(19) United States
(12) Patent Application Publication
(10) Pub. No.: US 2009/0099660 A1
(43) Pub. Date: Apr. 16, 2009

(54) Instrumentation to Facilitate Access into the Intervertebral Disc Space and Introduction of Materials Therein

(75) Inventors: Jeffrey L. Scifert, Arlington, TN (US); Scott D. Boden, Atlanta, GA (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 11/869,991

(22) Filed: Oct. 10, 2007

(51) Int. Cl. A61F 2/44; A61B 17/00; A61F 5/00; A61B 1/00; A61B 17/58; A61B 19/00

(52) U.S. Cl. 623/17.16; 606/80; 606/92; 128/898; 600/184; 606/90

(57) ABSTRACT

Methods for facilitating access to the intervertebral disc to deliver materials for disc repair are disclosed. The methods may be used to replace or augment nucleus pulposus as well as to perform interbody fusion procedures. These methods include providing access to the intervertebral disc space by drilling a channel through a vertebral bone, optionally accessing the intervertebral disc space to at least partially remove the disc tissue through the channel in the vertebrae; delivering a disc repairing material to the intervertebral disc space and back-filling the channel in the vertebrae with a channel sealing material. The methods may further comprise distracting the intervertebral height and inserting a restrictor into the channel in the vertebrae. Kits for practicing these methods are also disclosed.

### FIELD OF THE INVENTION

The present invention relates to methods and kits for treatment of intervertebral discs. More particularly, it discloses methods and kits for facilitating access to the intervertebral disc to deliver materials for disc repair.

### BACKGROUND OF THE INVENTION

As shown in FIG. 1, the spine 10 is composed of a column of vertebrae 12 that are individually separated from each other by intervertebral discs 16. The spinal cord 14 runs through the length of the spine 10. The discs 16 are an important part of the entire spinal column 10, and act like shock absorbers between adjacent vertebrae 12. The discs 16 must be able to absorb mechanical loads while simultaneously permitting constrained flexing of the spine 10.

Each disc 16 is shaped somewhat like a jelly donut, having a relatively soft inner region 17 surrounded by a strong, fibrous outer region 18. The gel-like inner region 17 is called the nucleus pulposus, and the reinforcing outer region 18 is called the annulus fibrosis. The nucleus pulposus 17 distributes mechanical loads placed upon the disc 16, while the annulus fibrosis 18 provides structural integrity and constrains the nucleus pulposus 17 to a specific spinal region.

Degenerated discs are a significant source of spine-related pain. As people age, the nucleus pulposus begins to dehydrate. Dehydrated disc have a very limited ability to absorb shock and are a significant source of spine-related pain. In addition, the annulus fibrosus may tear due to an injury or the aging process allowing the nucleus pulposus to extrude through the tear. This condition is known as disc herniation and is typically referred to as slipped disc, ruptured disc, or a bulging disc. It is very common for the herniated disc to press against spinal nerves located near the posterior side of each disc all along the spine, causing radiating pain, numbness, tingling, and diminished strength and/or range of motion. In addition, the contact of the inner nuclear gel, which contains inflammatory proteins, with a nerve can also cause significant pain.

Amongst sufferers of chronic pain, spine-related problems constitute the bulk of such complaints. Spinal pain has been estimated to exist in as much as 66% of the general population. Beyond the substantial discomfort that back pain inflicts upon individuals, spine-related pain also incurs heavy societal costs. For example, as many as one million spine surgeries, and as many as five million interventional procedures, are estimated to be performed in the United States each year. Well beyond the purely medical and psychological burdens imposed by such procedures, the subsequent social costs related to productivity, disability compensation and lost taxes are substantial.

In light of the foregoing, there is a need in the art for improving procedures and devices associated with performing spinal surgery.

### SUMMARY OF THE INVENTION

In one aspect, methods for treatment of intervertebral discs are provided. These methods comprise providing access to the intervertebral disc space by drilling a channel through a vertebral bone; (ii) delivering a disc repairing material to intervertebral disc space; and (iii) back-filling the channel in the vertebrae with a channel sealing material.

These methods may further comprise distracting the intervertebral height prior to introducing the non-fusion material, accessing the intervertebral disc to at least partially remove disc tissue and inserting a restrictor into the channel in the vertebrae.

In another aspect, a kit for treating an interverebral disc is provided. The kit comprises a restrictor, a disc tissue removing tool, a disc repairing material, and a disc repairing material delivery system. In some embodiments, the kit may also include a drilling instrument, a channel sealing material and the channel sealing material delivery system, a distraction tool, or any combinations thereof.

### BRIEF DESCRIPTION OF THE FIGURES

FIG. 1 is a perspective view of a portion of a spinal column.

FIG. 2 and FIG. 3 illustrate one possible embodiment of the pituitary rogeurs.

FIG. 4 illustrates one possible embodiment of the disc separating tool.

FIG. 5 illustrates one possible embodiment of the restrictor.

FIG. 6 shows one possible embodiment of the disc distracting tool.

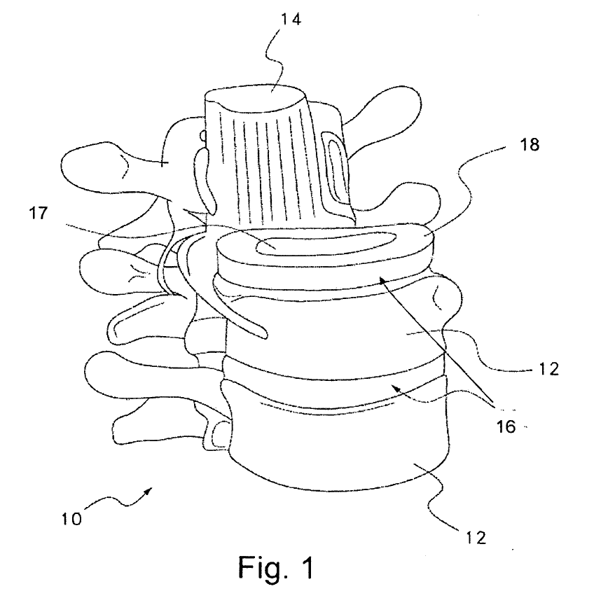

### DETAILED DESCRIPTION OF THE INVENTION

Unless defined otherwise, all technical and scientific terms used herein have the same meaning as is commonly understood by one of skill in the art to which this invention belongs. All publications and patents referred to herein are incorporated by reference in their entirety.

In one aspect, kits for treatment of intervertebral discs are provided. The kits comprise a restrictor, a disc tissue removing tool, a disc repairing material, and a disc repairing material delivery system. In some embodiments, the kit may also include a drilling instrument, a channel sealing material and the channel sealing material delivery system, a distraction tool, or any combinations thereof.

The drilling device is employed to create a channel in a vertebrae to access the intervertebral disc space. Suitable drilling devices include, but are not limited to, hand and motor powered drills, flexible drills, steerable drills, flexible burrs, and steerable burrs. In some embodiments, the drilling device may comprise a sharp pointed needle or a trocar needle that can be inserted within the cannula. One can also use curets or awls. Such devices are well known in the art and are disclosed, for example, in U.S. Pat. Nos. 7,141,074, 5,941,706, and 6,951,562, incorporated herein by reference in their entirety. Preferably, a steerable drilling device is used so the surgeon may select the best path through the vertebrae to the disc space.

In one embodiment, the physician uses minimally invasive techniques to access the vertebrae and to deliver the necessary equipment and materials to the verterbrae and into the disc space. In an alternative embodiment, the physician surgically opens the patient's back, cutting and retracting tissue until the vertebrae is exposed. With the exposed vertebrae, the physician can then drill into the vertebrae and access the disc space through the vertebrae.

To access the disc space, one may utilize drill a straight line through the vertebrae and end plate. Alternatively, one may be required to have a curve in the drilling path to cut through the end plate to access the disc. Such a curved path is disclosed in U.S. Pat. No. 6,805,697 to Helm, et al. For either embodiments, the channel is cut through the vertebrae and through the endplate to enter into the disc space, thereby leaving the annulus fibrosus intact.

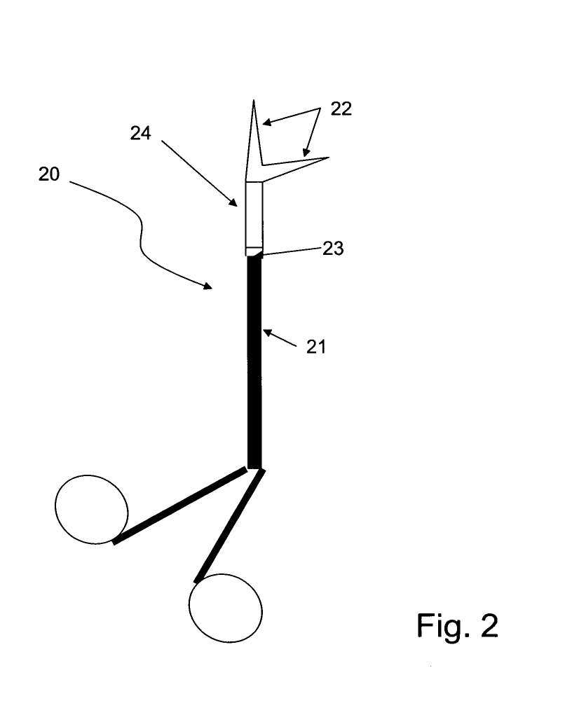

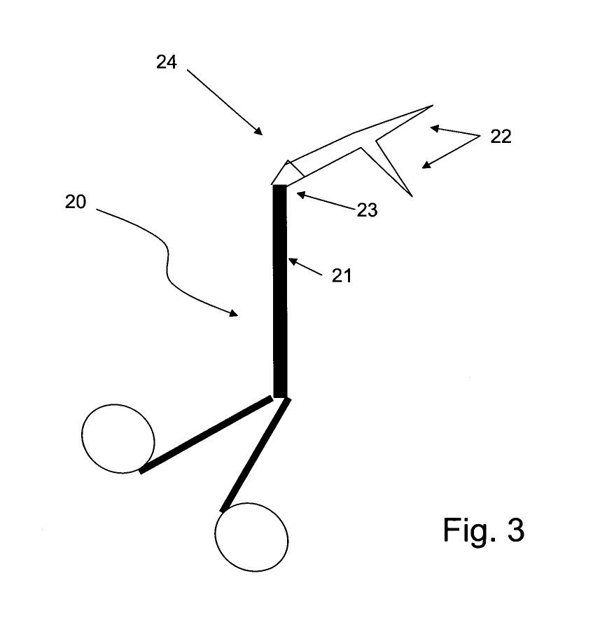

A disc tissue removing tool is used to fully or partially remove disc tissue. In some embodiments, the disc tissue removing tool may comprise a mechanical device such as, for example, pituitary rongeurs. Although the pituitary rongeurs may be used to remove entire disc, they also allow the surgeon to only remove certain parts of the disc tissue while leaving the rest of it in tact. Referring to FIG. 2, the pituitary rongeurs 20 may comprise a flexible shaft 21 with tips 22 disposed at a distal end of the shaft 21. The flexible shaft 21 enables the user to reach into remote parts of the disc space from the single access point. In addition, a rotating base 23 may also be inserted a short distance from the distal tip of the shaft 21 to enable the top portion 24 of the shaft 21 and the tips 22 to bend and rotate as shown in FIG. 3. Other types of standard surgical pituitary rongeurs, such as Micro Decker Pituitary Rongeurs (Life Instrument Corporation, Braintree, Mass.); Cushing Pituitary Rongeurs (Dixon Surgical Instruments, Wickford, Essex, UK); pituitary rongeurs (Codman Inc., Raynham, Mass.); and disc rongeurs (DePuv Spine, Inc., Raynham, Mass.) as well as mechanical instruments, such as, endoscopic scissors, scalpels, curettes, graspers, cutters, drills, microdebriders pituitary and the like may also be used.

In other embodiments, the disc tissue removing tool may comprise a disc separating device, especially when complete removal of the nucleus pulposus is desired. The disc separating device separates the nucleus pulposus from the annulus fibrosus by emulsifying the nucleus pulposus. The nucleus pulposus material may then be removed using any known irrigation or suction methods. One example of a suitable device for emulsifying of the nucleus pulposus is described in U.S. Patent Application No. 2006/0106410 to Serhan; Hassan A.; et al., incorporated herein by reference in its entirety. Optionally, irrigation and/or suction may be used to remove pieces of the nucleus pulposus.

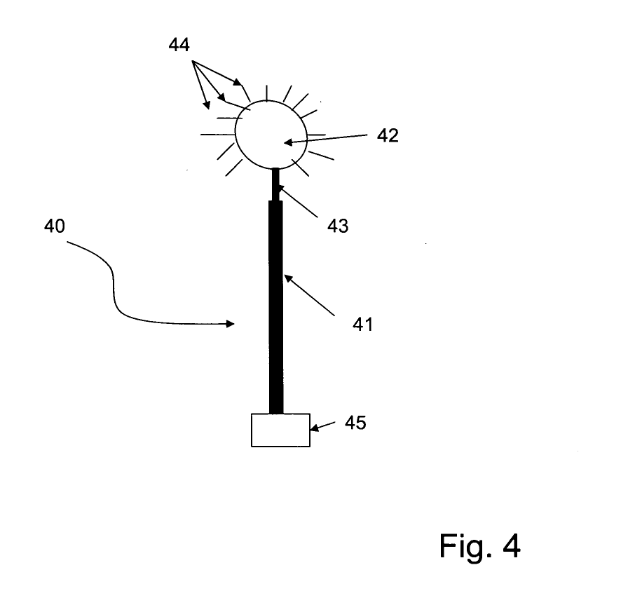

Another example of the disc separating device is illustrated in FIG. 4. The emulsifier 40 comprises a flexible shaft 41, a head 42 attached to the shaft 41 using a swivel connector 43 which enables the head 42 to rotate freely. A plurality of whips 44 are disposed around the head 42. In some embodiments, the whips may be retractable which may allow for easier movement of the device to and from the disc space. When the head 42 is rotated, the whips emulsify the nucleus pulposus, thus separating it from the disc. A motor 45 may be utilized to rotate the head 42. Alternatively, the head 42 may be rotated manually.

The disc tissue removing tool and the disc separating device are inserted through the channel into the disc space, again leaving the annulus fibrosus intact.

In some embodiments, a restrictor may be inserted into the channel in the vertebrae. The restrictor may be inserted anywhere within the channel, preferably near or at the end plate. One can insert the restrictor after drilling of the channel is completed, or after removal of the nucleus pulposus, or after insertion of disc repairing material. It may be preferable to insert the restrictor upon completion of drilling of the channel but before one removes the nucleus pulposus. The restrictor can prevent disc repairing material from leaving the disc space through the channel and prevents channel sealing material from entering into the disc space. The restrictor also can aid in the removal of the disc material via irrigation and/or suction by creating a barrier through which material cannot escape the disc space.

The restrictor may be formed from non-bioresorbable or bioresorbable, biocompatible polymers. Examples of such polymers include, but are not limited to, polyethylene, silicone, polyester, Nylon, Dacron, expanded polytetrafluroethylene (e-PTFE)polyamide collagen, polylactic acid (PLA) or polyglycolic acid (PGA), LPLA (poly(1-lactide)), DLPLA (poly(dl-lactide)), LPLA-DLPLA, PGA (polyglycolide), PGA-LPLA or PGA-DLPLA or combinations thereof.

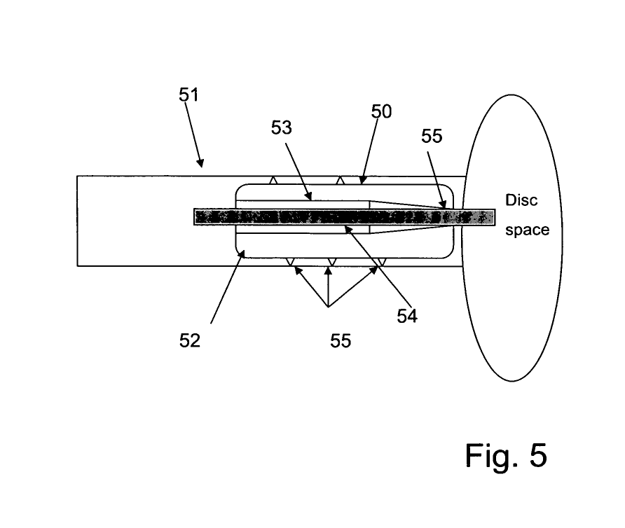

Referring to FIG. 5, in one embodiment, the restrictor 50 is shown inside a channel 51. The restrictor comprises a body 52 having a passageway 53 extending through the body 52. The passageway is sized to allow access of surgical devices 54 to and from the disc space while creating a tight seal between the restrictor and the device passing through the restrictor. In some embodiments, the restrictor may also comprise a resealable entry port 55, a check valve (not shown) or both that would ensure that the disc repairing material is contained within the disc space. The restrictor may also include anchoring mechanisms 55 such as, for example, threads, spines or similar to ensure that it stays in place throughout the procedure, and possibly afterwards. Alternatively, the restrictor may be held in place by friction such as when the diameter of the restrictor body is larger than the inner diameter of the bone channel. Non-limiting examples of restrictors include allograft bone, non-resorbable cement, resorbable cement, BIOSTOP G Resorbable Bone Cement Restrictor (Depuy Spine, Inc.), OrthoPrep™ Orthopedic—Cement Restrictors (Microtek Lab Inc., Carson, Calif.), Synplug cement restrictor (Isotis, Irvine, Calif.), and Buck Femoral Cement Restrictor (Smith & Nephew, London, England).

Disc distracting tools are used to pull the adjacent vertebras apart in order to maintain the disc height during the procedure. They are well known in the art. Some suitable examples of disc distracting devices are disclosed, for example, in U.S. Pat. No. 6,712,825 to Aebi, et al. and U.S. Patent Application No. 2006/0200138 to Michelson; Gary Karlin, which are incorporated herein by reference in its entirety.

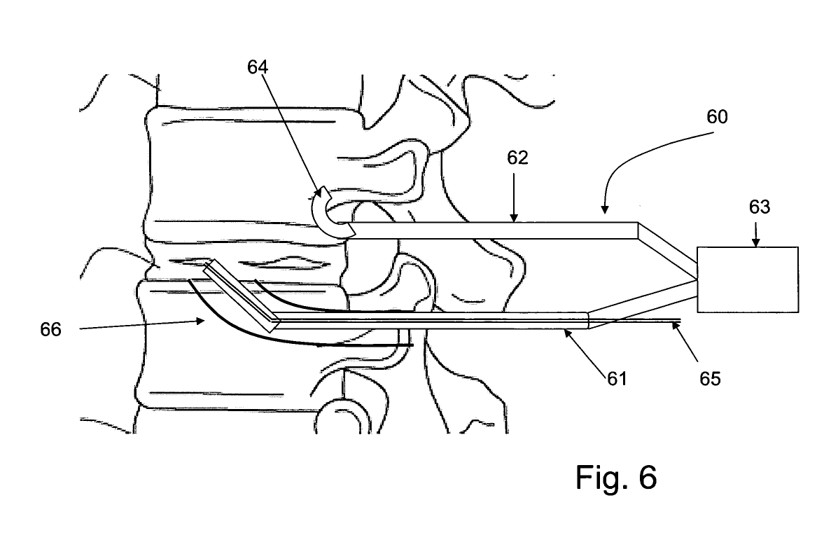

One suitable example of a disc distracting tool is also illustrated in FIG. 6. Referring to FIG. 6, the disc distracting tool 60 comprises a first arm 61 and a second arm 62. Distracting the vertebrae is achieved by moving the arms apart. In some embodiments, a mechanical gear mechanism 63 connected to the arms at their proximal ends may be used to spread the arms apart. Alternatively, the arms may be moved manually or using hydraulic means. In some embodiments, the arms of the disc distracting tool may be connected to a fulcrum point in order to provide for a parallel distraction of the disc.

In some embodiments, a hemispherical hook 64 may be disposed on distal end of either the first arm 61, the second arm 62, or both for improved anchoring of the arms to the vertebrae. During the distraction, the hook is placed under lamina if disposed on the first arm, and it is placed over the lamina, if disposed on the second arm. Alternatively, the first arm, the second arm or both may simply comprise a solid post. In other embodiments, one of the arms may be adopted to be inserted into the bone channel 66 and may extend into the disc space, as shown in FIG. 6. In this embodiment, that arm may include a lumen 65 that can be used to deliver disc repairing material to the disc space while the disc is being distracted.

The term “disc repairing material” means materials that are delivered to partially or fully replace, or augment the native disc materials. In various embodiments, the disc repairing material may include various additives that are described in detail below. These additives may be supplied in a separate container with the kit. They may be added to the disc repairing material as necessary by the surgeon before or during the procedure. Alternatively, some or all of these additives may be pre-mixed with the disc repairing materials at the time of kit manufacture.

The term “disc repairing material” includes both fusion and non-fusion disc repairing materials. In some instances, degenerated nucleus pulposus in the disc may be replaced with non-fusion disc repairing material, thus conserving the motion in the joint. Alternatively, the disc may be removed and adjacent vertebrae may be fused together by a procedure known as interbody fusion. In simplest terms, an interbody fusion means creating a solid bone bridge between adjacent vertebras which prevents the joint from moving, thus eliminating pain. Materials with different properties are employed in these procedures, namely non-fusion material for disc replacement and fusion material for interbody fusion.

The term “non-fusion disc repairing material” refers to material that are used when the movement in the intervertebral joint needs to be preserved. These materials are used to replace or augment nucleus pulposus to restore disc functions. The non-fusion disc repairing material preferably possess the same physical properties as the natural nucleus pulposus and cannot be absorbed or degraded by the body. On the other hand, the term “fusion disc repairing material” refers to material that are used when adjacent vertebras need to be fused. The fusion disc repairing materials provide scaffolding through and around which the patient's new bone will grow, gradually replacing these materials as the adjacent vertebrae fuse.

The non-fusion disc repairing material replaces the natural tissue and, thus, may preferably posses the same or similar properties as the natural nucleus pulposus. In one embodiment, a balloon or bag is placed through the channel into the disc. The non-fusion disc material is injected into the balloon or bag through an opening in the bag or balloon. After the balloon or bag is full, one can seal the opening to prevent leakage of the non-fusion disc material from the balloon or bag. In an alternative embodiment, the non-fusion disc material is inserted into the disc space directly, no balloon or bag is used.

The non-fusion disc material employed is preferably selected so the formed implant has sufficient load bearing capacity. In preferred embodiments, a compressive strength of at least about 0.1 Mpa is desired, although compressive strengths in the range of about 1 Mpa to about 20 Mpa are more preferred. In addition, the material may have other qualities that are important to non-fusion disc replacement including, but not limited to, mechanical strength, promotion of tissue formation, biocompatibility, sterilizability, minimal curing or setting time, optimum curing temperature, low viscosity for easy introduction into the disc space, and ability to withstand the large number of loading cycles experienced by the spine. It is preferable that the non-fusion disc replacement material not be biodegradability, however, biodegradability is an option.

A wide variety of biocompatible polymeric materials may be used. Such materials include, but are not limited to, elastic materials, such as elastomeric materials, hydrogels or other hydrophilic polymers, or composites thereof. Suitable elastomers include silicone, polyurethane, copolymers of silicone and polyurethane, polyolefins, such as polyisobutylene and polyisoprene, neoprene, nitrile, vulcanized rubber and combinations thereof. The vulcanized rubber described herein may be produced, for example, by a vulcanization process utilizing a copolymer produced as described, for example, in U.S. Pat. No. 5,245,098 to Summers et al. from 1-hexene and 5-methyl-1,4-hexadiene. Suitable hydrogels include natural hydrogels, and those formed from polyvinyl alcohol, acrylamides such as polyacrylic acid and poly(acrylonitrile-acrylic acid), non-resorbable polyurethanes, polyethylene glycol, poly(N-vinyl-2-pyrrolidone), acrylates such as polyacrylates, poly(2-hydroxy ethyl methacrylate), methyl methacrylate, 2-hydroxyethyl methacrylate, and copolymers of acrylates with N-vinyl pyrrolidone, N-vinyl lactams, acrylamide, polyurethanes and polyacrylonitrile, or may be other similar materials that form a hydrogel. The hydrogel materials may further be cross-linked to provide further strength to the implant. Examples of polyurethanes include thermoplastic polyurethanes, aliphatic polyurethanes, segmented polyurethanes, hydrophilic polyurethanes, polyether-urethane, polycarbonate-urethane and silicone polyether-urethane. Other suitable hydrophilic polymers include naturally-occurring materials such as glucomannan gel, polyphosphazenes, hyaluronic acid, polysaccharides, such as cross-linked carboxyl-containing polysaccharides, alkyl celluloses, hydroxyalkyl methyl celluloses, sodium chondroitin sulfate, cyclodextrin, polydextrose, dextran, gelatin, and combinations thereof.

Other suitable examples of the non-fusion disc repairing material include lightly cross-linked biocompatible homopolymers and copolymers of hydrophilic monomers such as 2-hydroxyalkyl acrylates and methacrylates, N-vinyl monomers, and ethylenically unsaturated acids and bases; polycyanoacrylate, polyethylene oxide-polypropylene glycol block copolymers, polygalacturonic acid, polyvinyl pyrrolidone, polyvinyl acetate, polyalkylene glycols, polyethylene oxide, collagen, sulfonated polymers, vinyl ether monomers or polymers, alginate, polyvinyl amines, polyvinyl pyridine, and polyvinyl imidazole. One can also use superabsorbent polymers (SAP) with or without additives. Superabsorbent polymers may include polymer chains that are synthetic, natural, and hybrid synthetic/natural polymers. Exemplary superabsorbent polymers may include, but are not limited to, polyacrylic acid, polymethacrylic acid, polymaleic acid, copolymers thereof, and alkali metal and ammonium salts thereof; graft copolymers of starch and acrylic acid, starch and saponified acrylonitrile, starch and saponified ethyl acrylate, and acrylate-vinyl acetate copolymers saponified; polyvinylpyrrolidone, polyvinyl alkylether, polyethylene oxide, polyacrylamide, and copolymers thereof; copolymers of maleic anhydride and alkyl vinylethers; saponified starch graft copolymers of acrylonitrile, acrylate esters, vinyl acetate, and starch graft copolymers of acrylic acid, methyacrylic acid, and maleic acid; the product of crosslinking acrylamide with backbones of kappa-carrageenan and soldium alginate using methylenebisacrylamide and potassium persulfate; and the product of crosslinking, using a bifunctional crosslinking reagent, an acyl-modified protein matrix such as soy protein isolate which has been acyl-modified by treatment with ethylenediaminetetraacetic acid dianhydride; mixtures and combinations thereof. Further, one can use silicone-based materials, polyethylene terephthalate, polycarbonate, thermoplastic elastomers and copolymers such as ether-ketone polymers such as poly(etheretherketone).

The fusion disc repairing material preferably promotes the bone growth between the vertebrae while ensuring stability of the spine before the fusion is complete. Examples of suitable fusion disc repairing material may include, but are not limited to, the MasterGraft® Matrix (Medtronic, Inc., Memphis, Tenn.); MasterGraft® Putty (Medtronic, Inc., Memphis, Tenn.); Absorbable Collagen Sponge (“ACS”) (Integra LifeSciences Corp., Plainsboro, N.J.); Collagraft® Bone Graft Matrix (Zimmer Holdings, Inc., Warsaw, Ind.); tricalcium phosphate granules e. g. , ChronOS® or Ceros® TCP (Mathys Ltd., Switzerland); Norian injectable cements (Norian Corp., Cupertino, Calif.); porous bone graft substitute, e.g., ProOsteon Implant 500® (Interpore Int., Irvine, Calif.); micro glass granules e.g., BiGran® (Orthovita, Malvern, Pa.); calcium phosphate e.g., Alpha BSM®, (ETEX Corp., Cambridge, Mass.); calcium phosphate-based bone cement e.g., BoneSource®, (Orthofix Inc., McKinney, Tex.); gel, putty and flex forms, e.g., Grafton DMB®, (Osteotech Inc., Eatontown, N.J.); artificial formable bone matrix marketed by Bioapatite AB, Sweden; bovine skin collagen fibers coated with hydroxyapatite, e. g. , Healos® (Johnson & Johnson, New Brunswick, N.J.); collagen sponges, e. g., Hemostagene® (Coletica SA, France), or e.g., Helisat® (Integra Life Sciences Inc.); bioresorbable polymer and bone cement, e.g., OrthoDyn (DynaGen Inc., Cambridge, Mass.); biodegradable POB/PBT copolymers marketed by IsoTis; biodegradable polymers, e. g. , Prolease® and Medisorb® (Alkermes, Cambridge, Mass.); bone chips (e.g., 30/70 cortical/cancellous); calcium aluminates; and hydrogels.

The fusion disc repairing material may also be injectable; examples of such injectable matrices include Norian® SRS® Bone Void Filler (Norian Corp.); CORTOSS® Injectable Synthetic Bone Filler (Orthovita); and Cerament Bone Void Filler (Bone Support AB, Sweden). Other materials that are suitable as matrices include polysaccharides, proteins and polypeptides, glycosaminoglycans, proteoglycans, collagen, elastin, hyaluronic acid, dermatan sulfate, chitin, chitosan, pectin, (modified) dextran, (modified) starch, or mixtures or composites thereof.

Any suitable bone cement including, but not limited to, acrylic based bone cement, pastes comprising bone particles, or ceramic based cements may be used. Preferably, a low-viscosity liquid bone cement is employed. Synthetic polymers may also be employed, including for example biodegradable synthetic polymers such as polylactic acid, polyglycolide, polylactic polyglycolic acid copolymers (“PLGA”), polycaprolactone (“PCL”), poly(dioxanone), poly(trimethylene carbonate) copolymers, polyglyconate, poly(propylene fumarate), poly(ethylene terephthalate), poly(butylene terephthalate), polyethyleneglycol, polycaprolactone copolymers, polyhydroxybutyrate, polyhydroxyvalerate, tyrosine-derived polycarbonates and any random or (multi-)block copolymers, such as bipolymer, terpolymer, quaterpolymer, etc., that can be polymerized from the monomers related to the previously-listed homo- and copolymers.

The fusion disc repairing material may also include additional additives that promote bone formation. Suitable additives may include, but are not limited to, demineralized bone, all collagen types (not just type I), insoluble collagen derivatives, bone morphogenetic proteins (BMPs) including BMP-2, BMP-3, BMP-4, BMP-5, BMP-6, BMP-7, BMP-8, BMP-9, BMP-10, BMP-11, BMP-12, BMP-13, BMP-14, BMP-15, BMP-16, BMP-17, and BMP-18; LIM mineralization proteins, transforming growth factor (TGF-beta), insulin-like growth factor proteins including IGF-1 and IGF-2, platelet derived growth factor (PDGF), fibroblast growth factor proteins (FGF), vascular endothelial growth factor (VEGF), epidermal growth factor (EGF), angiogenic agents, bone promoters, cytokines, interleukins, genetic material, genes encoding bone promoting action, cells containing genes encoding bone promoting action; hormones, growth hormones such as somatotropin; bone digestors and combinations thereof.

The disc repairing materials may include an imaging agent. The term “imaging agent” is defined as a substance that can be visualized by imaging techniques including radiography, MRI, PET or SPECT, CT, fluoroscopy, luminescence and any combination thereof.

Suitable non-limiting examples of an imaging agent may be a radiographic marker, such as for example, barium, calcium phosphate, and/or metal beads. In another embodiment, the imaging agent may comprise iodine-based contrast agents, such as, for example, iopamidol, commercially available as Isovue™ (Bracco Diagnostics Inc., Princeton, N.J.) or iodixanol, commercially available as Visipaque™ (Nyocomed, Inc., Princeton, N.J.), and gandolinium-based contrast agents, such as, for example, gadodiaminde, commercially available as Omniscan™ (available from GE Healthcare, Princeton, N.J.).

Other examples would include linkage of the imaging agent or radioisotope to a component of the hygroscopic agent. Examples of radioisotope would include ¹⁸F, ³H, ¹²⁴I, ¹²⁵I, ¹³¹I, ³⁵S, ¹⁴C, ¹¹C or a fluorescent molecule.

Radioisotopes may be attached to a component of the hygroscopic agent, such as the hygroscopic compound, by using a chelating agent, such as EDTA or DTPA, and detected by gamma counter, scintillation counter, PET scanning, or autoradiography. Other methods of labeling the marker are described, for example, in the U.S. Pat. App. No. 2005/0118165 and in Hunter et al., Nature 194:495 (1962); G. S. David et al., Biochemistry 13:1014-1021 (1974); D. Pain et al., J Immunol Meth 40:219-230 (1981); and H. Nygren, J. Histochem Cytochem. 30:407 (1982), all of which are incorporated by reference herein.

In other embodiments, the imaging agent is a fluorescent label. Common fluorescent labels include fluorescein, dansyl, phycoerythryn, phycocyanin, allophycocyanin, o-phtaldehyde, and fluorescamine. In yet other embodiments, the imaging agent may comprise a fluorescence-emitting metal such as, for example, ¹⁵²Eu⁺ or other lantanoids. The fluorescence-emitting metals can be attached to a component of the hygroscopic agent, such as the hygroscopic compound, by using metal-chelating groups such as EDTA or DTPA.

In another embodiment, since radioisotopes may have a limited half-life, the imaging agent may be added to the hygroscopic agent within a few hours prior to administration.

In some embodiments, the repairing materials may also include a biologically active agent. A “biologically active agent” is defined as an agent designed to achieve a medically useful end. Biologically active agent may comprise anti-inflammatory compounds both steroidal and non-steroidal, analgesics, antibiotic and antibacterial agents. Suitable non-limiting examples of steroidal anti-inflammatory compounds are corticosteroids such as hydrocortisone, cortisol, hydroxyltriamcinolone, alpha-methyl dexamethasone, dexamethasone-phosphate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluocinolone, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, and triamcinolone. Mixtures of the above steroidal anti-inflammatory compounds can also be used.

Non-limiting examples of non-steroidal anti-inflammatory compounds include nabumetone, celecoxib, etodolac, nimesulide, apasone, gold, oxicams, such as piroxicam, isoxicam, meloxicam, tenoxicam, sudoxicam, and CP-14,304; the salicylates, such as aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; the fenamates, such as mefenamic, meclofenamic, flufenamic, niflumic, and tolfenamic acids; the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and the pyrazoles, such as phenylbutazone, oxyphenbutazone, feprazone, azapropazone, and trimethazone.

The variety of compounds encompassed by the anti-inflammatory group of agents are well-known to those skilled in the art. For detailed disclosure of the chemical structure, synthesis, side effects, etc. of non-steroidal anti-inflammatory compounds, reference may be made to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology 1, R. A. Scherrer, et al., Academic Press, New York (1974), each incorporated herein by reference. Mixtures of these non-steroidal anti-inflammatory compounds may also be employed, as well as the pharmacologically acceptable salts and esters of these compounds.

In some embodiments, analgesics may also be included. Analgesics may comprise, without limitation, non-steroid anti-inflammatory drugs, non-limiting examples of which have been recited above. Further, analgesics also include other types of compounds, such as, for example, opioids (such as, for example, morphine and naloxone), local anaesthetics (such as, for example, lidocaine), glutamate receptor antagonists, α-adrenoreceptor agonists, adenosine, canabinoids, cholinergic and GABA receptors agonists, and different neuropeptides. A detailed discussion of different analgesics is provided in Sawynok et al., (2003) Pharmacological Reviews, 55:1-20, the content of which is incorporated herein by reference.

Suitable examples of antibiotics and antibacterial agents include, but are not limited to, amikacin, gentamicin, kanamycin, neomycin, netilmicin, paromomycin, streptomycin, tobramycin and apramycin, streptovaricins, rifamycins, amoxicillin, ampicillin azlocillin, carbenicillin, cloxacillin, dicloxacillin, flucloxacillin, mezlocillin, nafcillin, piperacillin, pivampicillin, ticarcillin, cefacetrile, cefadroxil, cefalexin, cefaloglycin cefalotin, cefapirin cefazolin, cefradine, cefaclor, ceforanide, cefotiam cefprozil, cefuroxime, cefdinir, cefditoren, cefixime cefmenoxime, cefoperazone cefotaxime, cefpiramide, cefpodoxime, ceftazidime, ceftibuten, ceftriaxone, cefepime, cefquinome, sulbactam, tazobactam, clavulanic acid, ampicillin/sulbactam (sultamicillin), co-amoxiclav and combinations thereof.

The nucleus augmentation material may be delivered into the disc space through the channel in the vertebrae using a nucleus augmentation material delivery system such as, for example, a pump, catheter, syringe, a minimal access port such as a cannula or funnel, expandable tube designs, or any other device which can deliver disc repairing material into the disc space. Suitable examples also include, but are not limited to, systems disclosed in U.S. Pat. Nos. 5,681,317; 6,547,432; 6,155,463; 6,086,594; 6,048,346; and 7,134,782 among many others, which are incorporated herein in their entirety. Of course, if a restrictor has been placed in the channel prior to delivery of the disc repairing material, the delivery of the disc repairing material should be in a manner that passes through the opening of the restrictor.

After the procedure is completed, the channel in the vertebrae may be closed by backfilling the channel with a channel sealing material. Any suitable bone cement may be used for this purpose including, but not limited to, acrylic based bone cement, pastes comprising bone particles, or ceramic based cements may be used. In addition, any of the fusion disc repairing materials described above may be used as the channel sealing material.

In some embodiments, the channel sealing material may include imaging agent, agents that promote bone growth, and bioactive agents. Some suitable non-limiting examples of these ingredients are provided above in regard to discussion of potential additives to the disc repairing materials. A person skilled in the art should be able to easily select the appropriate ingredients and their amounts based on his or her general knowledge and intended purpose for adding the additives to the bone cement. The additives may be pre-mixed with the channel sealing material at the time of manufacture of the material or be supplied in a separate container with the kit.

The channel sealing material may be delivered to the channel in the vertebrae using a channel sealing material delivery system. The channel sealing material delivery system may comprise any known delivery systems including, but not limited, to syringes, pipettes, pumps, catheters, cannulas, funnels, or combinations thereof. In addition, a number of systems have been developed mainly for delivering channel sealing materials. Suitable examples include, but are not limited to, systems disclosed in U.S. Patent Nos. 5,681,317; 6,547,432; 6,155,463; 6,086,594; 6,048,346; and 7,134,782 among many others, which are incorporated herein in their entirety. It may be preferable to leave the restrictor in the channel prior to delivery of the channel sealing material.

In some embodiments, the kit further comprises a set of instructions. The set of instructions preferably comprises information for safe and efficient use of the kit. A person of the ordinary skill in the art will appreciate that the set of instructions may be provided in any form including, without limitations, written, electronic, audio-recorded, video-recorded, and any combination thereof.

Furthermore, the kit may optionally include other such as, for example, items that are described below in regard to description of the methods for repairing intervertebral discs.

The kits described above provide the surgeon with many of the tools necessary to practice the methods for treatment of intervertebral discs. The term “treatment” refers to executing a protocol, which may include administering one or more drugs to a patient (human or otherwise), in an effort to alleviate signs or symptoms of the disease. Alleviation can occur prior to signs or symptoms of disease appearing, as well as after their appearance. Thus, “treating” or “treatment” includes “preventing” or “prevention” of the disease. In addition, “treating” or “treatment” does not require complete alleviation of signs or symptoms, does not require a cure, and specifically includes protocols which have only a marginal effect on the patient.

These methods include (i)providing access to the intervertebral disc space by drilling a channel through a vertebral bone and the end plate; (ii) delivering a disc repairing material to intervertebral disc space; and (iii) back-filling the channel in the vertebrae with a channel sealing material. These methods may further include steps of distracting the intervertebral height prior to introducing the disc repairing material, accessing the intervertebral disc to at least partially remove disc tissue and inserting a restrictor into the channel in the vertebrae.

Using any known medical imaging system, a target vertebrae, i.e., a vertebrae through which the access to the intervertebral disc in need of repair may be gained, is identified. The target vertebrae may be above or below the intervertebral disc in need of treatment, and preferably is adjacent to the disc. The target vertebrae may be accessed using various surgical approaches including, but not limited to, anterior, posterior, or lateral including anterolateral and posterolateral approaches. To access the target vertebrae, one may use minimally invasive surgical techniques or non-minimally invasive surgical techniques.

Using minimally invasive surgical techniques, an area of the patient's skin where the incision will be made is identified and surgically prepared. To reach the target vertebrae, an incision is made in a patient's skin, and a guide is inserted into the incision. With the help of the medical imaging system, the surgeon may steer the guide through the subcutaneous tissue to the target vertebrae. In some embodiments, the guide may comprise threads, hooks, or any other known anchoring mechanisms, so that upon reaching the target vertebrae, the guide may be removably attached to it.

In some embodiments, the guide comprises a solid guidewire, thus creating an over-the-wire tract between the skin incision and the target vertebrae. Alternatively, the guide may comprise an elongated lumen and the surgical instruments may be passed through the lumen to reach the target vertebrae and eventually the intervertebral disc space. Accordingly, the size of the lumen is selected depending on the size of the surgical instruments that the surgeon intends to use during the procedure. In embodiments where the guide comprises the elongated lumen, it may be preferable to use an obdurator inserted into the lumen to steer the guide to the target vertebrae. Obdurators are specifically designed to penetrate, separate or manipulate soft tissue without inflicting damage to them.

A drilling device may then be advanced along the guidewire or through the elongated lumen from the skin incision toward the target vertebrae. The drilling device is utilized to remove bone from the target vertebrae to create a channel through the vertebrae into the disc space. The access to the disc space may be obtained through various parts of the vertebrae, but preferably through the end plate. The choice of the optimal path through the vertebrae the disc space is typically selected based on the surgical approach. For example, if a posterior surgical approach is used, the access to the disc space may be gained by drilling through the vertebral pedicle. On the other hand, drilling through the lateral or anterolateral vertebral body may be preferred when using anterior or anterolateral surgical approach.

A surgeon may employ medical imaging techniques to control and steer the drilling device. Accordingly, in some embodiments, a radiopaque marker may be provided on the distal end of the drilling device. Alternatively, a radiopaque contrast solution may be injected into the channel during drilling. Continuous observation of the drilling procedure ensures the surgeon's efficiency and the patient's safety.

To remove bone debris produced by drilling, in some embodiments, the drilling device may include a lumen where the distal end of the lumen is positioned adjacent to the cutting surface of the drilling device and the proximal end of the lumen is attached to a suction device or an irrigation device. Such drilling devices are well-known and are disclosed, for example, in U.S. Pat. No. 7,247,161 to Johnston, et al., incorporated herein by reference in its entirety. In other embodiments, the drilling device may be periodically withdrawn from the channel in the vertebrae and a suction or an irrigation device may be utilized to remove the debris. One example of the suitable suction and irrigation device is disclosed in U.S. Pat. No. 6,932,788 to Kamiyama, et al., incorporated herein by reference in its entirety.

Once the drilling is complete and the debris are removed, the channel through the vertebrae may be used to transport materials and surgical tools to and from the disc space. One may optionally line the channel with a guide tube.

Depending on the extent of the damage to the disc and the type of procedure, the disc tissue may be partially or fully removed. The term “disc tissue” includes both the nucleus pulposus tissue and the annulus fibrosis tissue and may refer to either one of these tissues. In some embodiments, however, the disc tissue may be left fully intact and only augmented with the disc repairing material. For some embodiments, only the nucleus pulposus is removed, leaving the annulus fibrosus intact.

A restrictor may be placed into the channel right after the channel is completed. In this embodiment, the restrictor would have an opening through which all the surgical instruments must pass before being introduced into the disc space. Alternatively, the restrictor may be placed into the channel immediately prior to administering of the disc repairing materials to the disc space. In some embodiments, the restrictor may be inserted into the channel only after the disc repairing materials are already administered.

A restrictor may also be utilized to contain the disc repairing material inside the disc space. Even if the restrictor is used, the channel in the vertebrae may still be backfilled after the completion of the procedure to promote bone regeneration in the vertebrae.

A person with ordinary skill in the art would undoubtedly be able to determine the type and the extent of the disc tissue that needs to be removed. For example, in an interbody fusion procedure at least some, or optionally all, of the annulus fibrosus needs to be removed to make sure that the new bone connects to vertebrae on both sides of the disc. The nucleus pulposus is obsolete once the spine is fused.

On the contrary, in disc replacement procedure, it is important to minimize, and preferably to eliminate, any damage to annulus fibrosus. Tears in annulus fibrosus may allow the non-fusion disc repairing material to leak out through the tears. Damage to the annulus fibrosus may result in the annulus fibrosus bulging out because of weaknesses in its walls. Thus, it is preferable to use non-fusion disc repairing material only in patients with an intact annulus fibrosus.

The disc removing tool(s) are inserted through the channel and the restrictor (if it has already been placed in the channel) to enter the disc space. One may use the pituitary rongeurs to remove portions or all of the nucleus pulposus. One may also use a disc separating device to remove portions or all of the nucleus pulposus. Irrigation and/or suction may optionally be used to help remove the disc material. These devices are described above.

One of the factors that effects functionality of the spine is the height of the spinal disc. Accordingly, before administering the disc repairing material to the disc space, the vertebras adjacent to the disc to be repaired or replaced optionally may be distracted using the disc distracting tool. This ensures that the proper disc height is maintained. One arm of the disc distracting tool may be placed in the channel. The other arm may be placed on the adjacent vertebrae using minimally invasive surgical techniques described above. The amount of distraction will be determined by the amount of reduction in height of the disc.

Next, the disc repairing material may be delivered into the disc space via the channel, and through the restrictor, if the restrictor has already been placed in the channel. In some embodiments, the disc repairing material may be injected directly into the disc space. It is preferable that the non-fusion disc repairing material is delivered under minimal pressure, less than 25 to 30 psi, so that the annulus fibrosus remains intact. In other embodiments, a flexible biocompatible bag such as a balloon may be delivered through the channel to the inside of the disc and then filled with the non-fusion disc repairing material. Medical imaging technology facilitates optimal delivery of the disc repairing material to the disc space. This technology enables one to verify the placement or the distribution of the disc repairing material within the disc space and the place of the balloon, if it is used. Accordingly, preferably the surgeon monitors delivery of the disc repairing material to the disc space using any known medical imaging system. If a balloon is used, one should seal the balloon after the disc repairing material has been placed inside the balloon.

In some embodiments, an interfusion device such cages, screws, or rods may be inserted between the vertebrae to facilitate fusion while adding strength and stability to the spine. In other embodiment, strong and stable interbody fusions may be achieved without the employment of interbody devices using only fusion disc repairing material as described above.

Depending on the type of material used, the disc repairing material may be contained inside the disc space without any further action from the surgeon. For example, a more viscous in-situ curing material is less likely to escape from disc space. Alternatively, the restrictor placed in the channel would prevent the disc repairing material from exiting the disc space through the channel, regardless of the type of material used. Of course, in embodiments where a balloon is employed, sealing the bag would ensure that the disc repairing material is contained inside the disc space.

To help repair the vertebrae, one should backfill the channel with the channel sealing material. Preferably, a low-viscosity to medium viscosity material is employed to allow for delivery through a needle or minimally invasive port, thus avoiding extensive surgical intervention. Backfilling of the channel may be observed using standard medical imaging techniques.

Preferably, the channel sealing material is prevented from entering the disc space. The restrictor can act as a barrier to prevent the channel sealing material from entering the disc space.

Finally, the guide may be removed from the patient and the incision in the patient's skin may be closed using any known surgical technique.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1. A method for treating an interverebral disc comprising:
(i) providing access to an intervertebral disc space by drilling a channel through a vertebral bone;
(ii) delivering a disc repairing material to the intervertebral disc space; and
(iii) back-filling the channel in the vertebrae with a channel sealing material.

2. The method of claim 1 wherein the vertebral bone is selected from the group comprising vertebral body, vertebral pedicle, and endplate.

3. The method of claim 1, wherein the access to the disc space is provided by drilling through a vertebral pedicle using a posterior surgical approach.

4. The method of claim 1, wherein the access to the disc space is provided by drilling through an anterolateral vertebral body using anterior or lateral surgical approaches.

5. The method of claim 1 further comprising accessing the intervertebral disc space to at least partially remove disc tissue.

6. The method of claim 5 further comprising placing a restrictor at the distal end of the channel after drilling the channel.

7. The method of claim 1 further comprising inserting a restrictor comprising a check valve into the channel in the vertebrae.

8. The method of claim 7 wherein the restrictor is inserted into the channel in the vertebrae prior to the step of delivering a disc repairing material to intervertebral disc space and wherein the disc repairing material is delivered through the restrictor.

9. The method of claim 1 wherein the step of delivering a disc repairing material to the intervertebral disc space comprising delivering a non-fusion disc repairing material.

10. The method of claim 1 wherein the step of delivering a disc repairing material to the intervertebral disc space comprising delivering a fusion disc repairing material.

11. The method of claim 1 further comprising distracting the vertebrae adjacent to the intervertebral disc prior to introducing the disc repairing material.

12. The method of claim 11, wherein the distraction step uses a distraction tool comprising two arms wherein one arm of the distraction tool is a laminar hook and the other arm is placed inside the bone channel.

13. The method of claim 12, wherein the arms of the distraction tool are connected to a gearbox mechanism or a distraction fulcrum point which provides for parallel distraction of the vertebral bodies.

14. The method of claim 1 wherein the step of delivering a disc repairing material to intervertebral disc space comprises delivering a balloon to the disc space and filling the balloon with the disc repairing material.

15. A method for providing access to a disc space through a bone channel comprising drilling the bone channel through a vertebra and placing a restrictor into the channel wherein the restrictor comprises a check valve.

16. A system for nucleus replacement in a disc comprising:
a rotating steerable drill for forming a channel by drilling through the vertebral bone into the intervertebral disc space; and
a restrictor comprising a check valve to be placed within the channel wherein the restrictor allows delivery of a disc repairing material to the disc space.

17. A kit for treating an intervertebral disc comprising:
a drilling instrument;
a restrictor;
a disc tissue removing tool;
a disc repairing material; and
a disc repairing material delivery system.

18. The kit of claim 17 further comprising a channel sealing material and a channel sealing material delivery system.

19. The kit of claim 18, wherein the restrictor comprises a one-way check valve.

20. The kit of claim 18, wherein the a disc tissue removing tool is selected from the group consisting of pituitary rongeurs, endoscopic scissors, scalpels, curettes, graspers, cutters, drills, microdebriders, and disc separating devices.

21. The kit of claim 18 further comprising a distraction tool.

22. The kit of claim 21 wherein the distraction tool comprises two arms wherein one arm of the distraction tool is a laminar hook and the other arm is placed inside the bone channel.

23. The kit of claim 21 wherein the distraction tool comprises two arms connected to a gearbox mechanism or a distraction fulcrum point.
